(19) United States
(12) Patent Application Publication
(10) Pub. No.: US 2004/0023228 A1
(43) Pub. Date: Feb. 5, 2004

(54) System and method for analysing active ingredients designed to influence intra-cellular processes

(76) Inventors: Georg Bohnert, Gegenbach (DE); Juan Camacho-Gomez, Jena (DE); Steffen Kirchstein, Zollnitz (DE); Leonhard Kittler, Jena (DE); Tobias Neumann, Jena (DE); Eberhard Unger, Cospeda (DE)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/221,953

(22) PCT Filed: Mar. 15, 2001
(86) PCT No.: PCT/EP01/02922

(30) Foreign Application Priority Data
Mar. 17, 2000 (DE) 100 13 854.3

(51) Int. Cl. C12Q 1/68; G01N 33/53

(52) U.S. Cl. 435/6; 435/7.1

(57) ABSTRACT

Method for examining active ingredients for influencing intracellular processes, wherein at least one active ingredient is added to an assembly equilibrium of proteins capable of assembly and a first fluorescence correlation measurement (FCS) is carried out after the addition of fluorescence-labeled monomers and/or dimers.

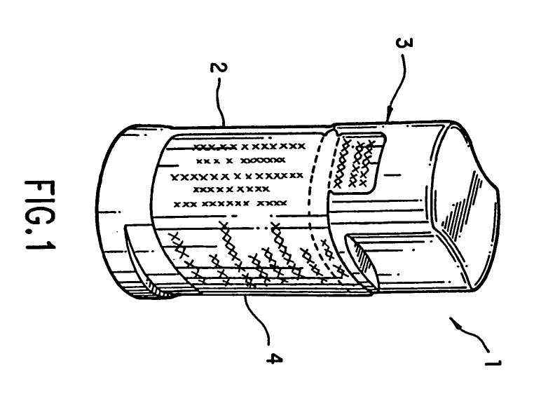

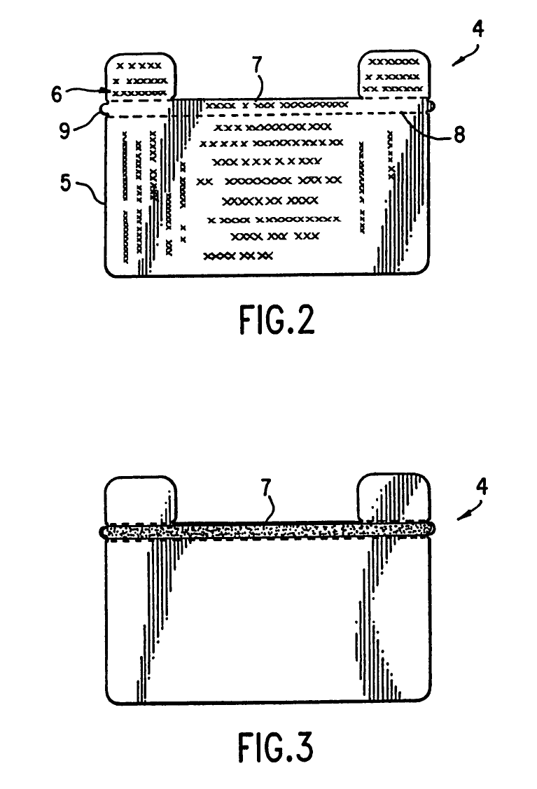

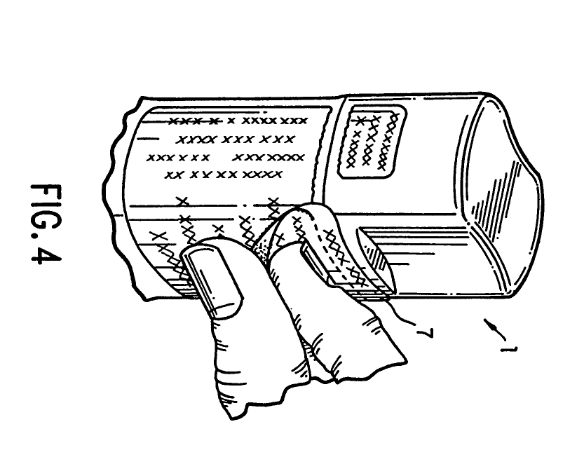

### CROSS-REFERENCE TO RELATED APPLICATIONS

This application claims priority of PCT Application Serial No. PCT/EP01/02922, filed Mar. 15, 2001 and German Application No. 100 13 854.3, filed Mar. 17, 2000, the complete disclosures of which are hereby incorporated by reference.

### BACKGROUND OF THE INVENTION

a) Field of the Invention

Arrangement and method for examining active ingredients for influencing intracellular processes.

b) Problem Addressed by the Invention

Active ingredients which limit the viability of cells by interfering with the assembly equilibrium or assembly dynamic of proteins and protein complexes can be detected by previously used test methods. However, these methods can be carried out only with high, and therefore pharmacologically irrelevant, concentrations of active ingredients or very laboriously with pharmacologically relevant concentrations of active ingredients or, for example, based on the use of radioactively labeled isotopes.

b) Solution of the Problem

Active ingredients such as paclitaxel or vinblastin which limit the viability of cells by interfering with the assembly equilibrium or assembly dynamic of proteins and protein complexes can be detected by means of a novel FCS-based screening method. The method is accordingly suitable for contributing to the development of cancerostatics and cytocides (fungicides, herbicides, etc.).

The binding of fluorescence-labeled proteins to other proteins in solutions can be measured by means of fluorescence correlation spectroscopy (FCS) either

a) by fluorescence labeling of both proteins with different labeling or

b) by the binding of a fluorescence-labeled protein to an unlabeled protein which is appreciably larger.

In the first case a), the binding of the two proteins is detected by simultaneous detection of the two fluorescence labels in the focus volume of the microscope.

In the second case b), the binding is detected by the difference in the diffusion rate between the fluorescence-labeled protein not bound to the appreciably larger partner protein, which has a high diffusion rate, and the fluorescence-labeled protein bound to the appreciably larger partner protein, which has an appreciably lower and therefore distinguishable diffusion rate.

FCS is a modern measurement method. Fluorescence events starting from individual molecules are recorded and statistically evaluated by a correlation analysis. The diffusion rate can be determined from these combined statistics allowing conclusions to be drawn about the size and binding behavior of molecular-biological reactions. FCS is carried out by confocal imaging with CW laser excitation. Individual molecules diffuse through the detection volume (femtoliter) defined in this way and a photon shower is emitted through the fluorescence label which is recorded by an avalanche diode. The photon showers can only be differentiated when the concentration of fluorescing molecules is less than 10⁻⁸M, so that this method can be used to examine small molecular amounts to the picomol range.

Proteins that are capable of assembly such as tubulin, actin, and tau protein are an indispensable component of cells. The equilibrium of these proteins between their monomeric, oligomeric or other polymeric form are a necessary precondition for life.

The invention is based on influencing exchange kinetics in the assembly equilibrium of proteins and protein complexes, the disruption of these exchange kinetics, e.g., by active ingredients, and the detection of these disruptions. It is possible to detect influences of active ingredients in concentration ranges close to pharmacologically achievable conditions. The special advance made by this method consists in focusing on minimal equilibrium changes through very low active ingredient concentrations instead of the usual influencing of the assembly of proteins and protein complexes by high concentrations of active ingredients which can not be achieved pharmacologically.

This invention is especially valuable in that it overcomes the difficulties associated with the need for use of fast assays with excessively high concentrations of active ingredients or pharmacologically relevant concentrations of active ingredients in complicated and expensive assays not suited for high throughput screening (HTS).

This test can be carried out in microtiter format and is accordingly suited for HTS in large substance libraries.

### BRIEF DESCRIPTION OF THE ILLUSTRATIONS

In the illustrations: Illustration 1a is a graph showing the decrease in the proportion of the “fast” dimers with reference to the decrease;

Illustration 1b shows the increase in slow polymers before reach an equilibrium;

Illustration 2 shows the time dependent decrease in the proportion of fast dimers with the addition of an active ingredient; and

Illustration 3 shows the decrease rate of various active ingredients compared to the decrease rate without active ingredients.

### DESCRIPTION OF PREFERRED EXAMPLES AND EMBODIMENTS

An example for the search of substances affecting polymer-oligomer equilibrium kinetics is the incorporation of rhodamine-labeled tubulin which is commercially available, e.g., from Cytoskeleton, Item No. T331/M, in preformed microtubles under quasi-physiologic conditions.

The incorporation depends on the equilibrium and exchange kinetics between polymers (microtubule) and dimers.

For assembly of the microtubules, a solution of about 1 mg/ml tubulin (about 10⁻⁵M) is produced in a known manner, e.g., according to Shelanski et al.: M. L. Shelanski, F. Gaskin and C. R. Cantor, 1973, “Microtubule assembly in the absence of added nucleotides”, Proc Natl Acad Sci USA 70, 765-768.

The assembly is carried out, for example, in a buffer of the following composition:

| | | | | |
|---|---|---|---|---|
| 20 mM Pipes | 12.096 | g | | |
| 1 mM EGTA | 0.76 | g | | |
| 80 mM NaCl | 9.35 | g | | |
| 0.5 mM MgCl₂ | 0.0952 | g | MgCl₂ × 6 H₂O | 0.2033 g |
| 1 mM DTT | 0.308 | g | | |

to 2 1 distilled water, pH 6.8

or other generally known test buffers for tubulin assembly pH 5.8-9.5 at the physiological temperature of 37° C. with the addition of GTP (guanosine triphosphate) as energy source, for example, in a vessel. In this connection, an equilibrium takes place between microtubules (polymers) and the tubulin dimer (mol mass 110 kDa) within approximately 15 to 20 minutes.

This equilibrium is dynamic and is characterized by a constant exchange of tubulin dimers between polymer and dissolved form.

A small amount (10⁻⁹M) of rhodamine-labeled tubulin (or other fluorescence-labeled tubulin dimer) dissolved in the tubulin assembly buffer described above is now added to a solution of the adjusted equilibrium between microtubules and tubulin dimers.

By pipetting in a suitable vessel, for example, in a microtiter plate, a fluorescence correlation measurement FCS (Carl Zeiss: Confocor) is carried out and the curve of the diffusion time is determined.

In order to achieve the adjusted temperature, a temperature regulating plate having a recess for the optical path of the microscope is provided under the specimen vessel.

At the start of the measurement, only fluorescence-labeled tubulin dimers with a fast diffusion constant are present and detectable in the solution. Over the course of time, the incorporation of the fluorescence-labeled tubulin dimers in the polymers can be tracked by FCS, wherein an equilibrium adjustment is brought about between the fluorescence-labeled polymer and dimer.

Illustration 1a shows the decrease in the proportion of “fast” dimers with reference to the decrease in diffusion time. Illustration 1b shows the increase in “slow” polymers before reaching an equilibrium.

Active ingredients such as paclitaxel or vinblastin, both of which are used against cancer in humans, are capable of hindering this exchange between tubulin dimers and polymers in substoichiometric ratios.

Thus, the pharmacologically relevant potential of the above-mentioned active ingredients consists not in preventing or shifting the adjustment of the equilibrium between protein polymers and protein dimers in clearly substoichiometric concentrations, but rather in completely or partially hindering the dynamic exchange in the equilibrium. This is a matter of influencing the dynamic instability of the microtubules. However, this characteristic of the microtubules and of other proteins capable of assembly is a precondition for the life of the cells.

Cancer cells with their increased metabolism require flexibility of the microtubules more than somatic cells, which is a reason for the relatively selective action of the above-mentioned active ingredients against cancer cells.

When the test (assembly, buffer) described above is repeated and the assembly mixture of 10⁻⁵M tubulin with active ingredients such as 10⁻⁸M to 10⁻¹¹M paclitaxel or 10⁻⁸M to 10⁻⁹M vinblastin is added, microtubules occur again which are in equilibrium with unpolymerized tubulin dimers.

It is particularly advantageous that the active ingredient can be added in pharmacologically relevant concentrations. The action of substances with a known effect on the described kinetics (nocodazole, taxol) can be tracked up to concentration of 10⁻¹¹ M.

A small amount of 10⁻⁹M rhodamine-labeled tubulin (or other fluorescence-labeled tubulin), for example, dissolved in the tubulin assembly buffer described above is added to this solution of the adjusted equilibrium between microtubules and tubulin dimers.

After pipetting in a suitable specimen vessel, for example, in a microtiter plate, an FCS measurement is carried out again.

At the start of measurement, only fluorescence-labeled tubulin dimers with a fast diffusion constant are present in the solution and are detectable based on the diffusion time.

Over the course of time, the incorporation of the fluorescence-labeled tubulin dimers in the polymers can be tracked by means of FCS. A delay in the equilibrium adjustment between fluorescence-labeled polymer and dimer can be brought about by the active ingredients used.

Illustration 2 shows the time-dependent decrease in the proportion of fast dimers with the addition of an active ingredient (top) compared to the previously measured (Illustration 1) decrease without active ingredients (bottom).

Illustration 3 shows the decrease rate of various active ingredients compared to the decrease rate without active ingredients as was explained with reference to Illustration 1.

It is clear that the action of test substances on the exchange rates in the assembly equilibrium of proteins capable of assembly is detected in a simple test system.

A test system of the kind described above can advantageously be carried out by modifying a Confocor inverted FCS microscope by means of an X/Y table for controlling different specimen vessels.

A temperature regulating plate which maintains the adjusted temperature of the assembly equilibrium and which has an opening for the measurement beam to pass through can advantageously be provided under the X/Y table.

The specimen vessels can be assembled in a microtiter plate (MTP) and the pipetting and subsequent measurement without the addition of active ingredients can be carried out in an opening of the MTP and the measurement with added active ingredient can be carried out in additional openings.

There are two possibilities for time-dependent measurement: Either the curve is detemined over 5 to 10 minutes, for example, for every specimen or a first value is determined by scanning different specimens and at least a second value is determined in at least a second scanning step and a time curve is determined in this way.

The measured value or curve of measured values for measurement without active ingredients is stored and compared with measured values or value curves with the addition of active ingredients.

In this way, the influence of various active ingredients on the dynamic exchange with the addition of fluorescence-labeled substances can be determined and the action of the active ingredients on cancer cells can be assessed.

The test system described above is suited for HTS and is reduced to a simple yes/no decision with respect to the influence of the exchange rates between protein and protein assemblage. At the same time, it is robust and makes do with small quantities of proteins.

The following are additional proteins that are capable of assembly for generating an assembly equilibrium:

1. Actin:

In general, actin is prepared at −80° C. Actin is thawed in a water bath (37° C.) from −80° C. and, immediately after thawing, the actin is placed in an ice bath or refrigerator at 4° C. A buffer is used to dilute it to a concentration of 1 mg/ml and it is allowed to stand for at least 1 hour. It is then stored at refrigerator temperature for 12 hours. The final concentration of 250 nM to 2.4 μm is then adjusted.

2. Tau Protein:

David M. Wilson and Lester 1. Binder, “Polymerization of Microtubule-associated Protein Tau under Near-physiological Conditions”, The Journal of Biological Chemistry, Vol. 270, No. 41; pp. 24306-24314, 1995 Conditions for tau polymerization: In general, tau protein is prepared at −80° C. It is then thawed at 4° C., diluted with 10 mM Tris pH 7.2 containing DTT or β-Mercaptoethanol and incubated at 37° C. The final protein concentration is 1 to 10 μM. When using buffers with pH of less than 7, 100 mM MES is used.

Comments:

MES: β-Morpholino-ethanesulfonic acid

Tris: Tris (hydroxymethyl) aminomethane

DTT: Dithiothreitol

With 1 and 2., ATP (adenosin triphosphate) can also be used in addition to GTP as energy source.

While the foregoing description and drawings represent the present invention, it will be obvious to those skilled in the art that various changes may be made therein without departing from the true spirit and scope of the present invention.

20. (new) the method according to claim 17, wherein the first FCS measurement is repeated for different active ingredients or active ingredient combinations and is compared with the second FCS measurement.

21. (New) The method according to claim 18, wherein the time-dependent curve of the diffusion time is determined in the first and second FCS measurement.

22. (New) The method according to at claim 17, wherein a time-dependent curve is determined during the time-dependent determination by means of at least the first FCS measurement with different specimens successively.

23. (New) The method according to claim 17, wherein a first value is determined initially for the specimens by scanning different specimens and at least one additional value is determined for the specimens after scanning additional specimens.

24. (New) The method according to claim 17, wherein the measurement is carried out after the addition of the fluorescence-labeled substances.

25. (New) The method according to claim 17, wherein active ingredients such as paclitaxel, nocodazole, vinblastin, colchicine are examined.

26. (New) The method according to claim 17, wherein proteins such as tubulin, F-actin or tau protein are used as proteins capable of assembly.

27. (New) An arrangement for the examination of active ingredients which are provided for influencing intracellular processes, comprising:
a fluorescence correlation spectroscopy system; and
means for carrying out a first fluorescence correlation measurement using said system of an assembly equilibrium of proteins that are capable of assembly by adding at least one active ingredient and fluorescence-labeled monomers and/or dimers.

28. (New) The arrangement according to claim 27, wherein a second FCS measurement of an assembly equilibrium is carried out by adding fluorescence-labeled monomers and/or dimers without active ingredients and the measurement values of the first and second measurements are compared.

29. (New) The arrangement according to claim 27, wherein said system is a microscopic arrangement for FCS measurement wherein specimen vessels in which the substances are pipetted are detected.

30. (New) The arrangement according to claim 27, wherein said microscopic arrangement is an inverted microscope.

31. (New) The arrangement according to claim 27, wherein the time-dependent curve of the diffusion time is determined in the FCS measurement.

32. (New) The arrangement according to claim 27, wherein an X/Y displacement unit is provided for detecting different specimen vessels.

33. (New) The arrangement according to claim 27, wherein the specimen vessels are temperature-regulated.
